(19) United States
(12) Patent Application Publication
(10) Pub. No.: US 2002/0102221 A1
(43) Pub. Date: Aug. 1, 2002

(54) Liquid dentistry

(76) Inventor: Douglas Brent Harwood, Port Townsend, WA (US)

Correspondence Address: Douglas Brent Harwood, 5614 Cape George Road, Port Townsend, WA, 98368, US

(21) Appl. No.: 09/773,081

(22) Filed: Feb. 1, 2001

(51) Int. Cl. A61K 7/16; A61K 35/78

(52) U.S. Cl. 424/49; 424/725.1

(57) ABSTRACT

By discovering the process by which tooth tartar can be disolved I can only speculate as to how this solution will be used to help solve the universal problem of tartar build up and decay.

Freshly Activated Carbon Tartar—Removal FRACT-R process provides an unintrusive therby painfree and highly effective removal through a process that could narratively be termed Liquid Detistry.

The implications for dental care and hygiene are stunning if this is accepted as a new art.

### CROSS REFERENCE TO RELATED APPLICATIONS

None Known

### STATEMENT REGARDING FEDERALLY SPONSERED RESEARCH

There has been no federally funded research.

### REFERENCE TO A MICROFICH APPENDIX

None known.

### BACKGROUND OF THE INVENTION

Dentistry.

To which the applicant claims no more insight other than what is commonly known about the practice. Facts about the essential aspects and goals of dental proceedures have become revealed through this new process which give insights as to what is wrong about “solid” dentistry. The most basic premise challenge revealed by this process asserts that tooth tartar can be liquified and removed without drilling, grinding metal tools, or scraping, thus challenging the prior art of tooth tartar removal.

### BRIEF SUMMARY OF THE INVENTION

A patent is sought for the discovery, and the proceedures and various techniques which follow from and are allowed by this discovery, that tooth tartar can be safely dissolved in the mouth.

The scope of the patent sought hopes to include all manner of devise and contrivance which might come about to employ, facilatate, or embellish the process which is derived through this discovery.

It is a simple but far reaching claim to say that I, Douglas Brent Harwood, did discover and invent a precise, specific process to dissolve and remove tooth tartar from teeth inside the mouth, gently, painlessly, antisepticly pure, without anesthetic.

### DETAILED DESCRIPTION OF THE INVENTION

The Discovery: On Jan. 10, 2001, I (DBH) discovered that freshly activated charcoal will dissolve the substance tooth tartar.

All the processes and techniques for the actual removal of the tartar stem entirely from this discovery, that tooth tartar can be liquified in the mouth. Hence actual technique and tool usage may vary according to how this discovery is used. Nonetheless the precise process of applying freshly activated charcoal, while varied in how it is achieved, i.e. what tools are used, it will be achieved only through the application of freshly activated charcoal. Here is described the process, and analysis.

Freshly Activated Charcoal Tooth Tartar Liquidification and Removal

This process is unbelieveably simple and pain free. It will surprise anyone who experiences it. Because of the incredible hardness and adhesiveness of tooth tartar and its multilayered filmlike structure the FACT-R (Freshly Activated Charcoal Tartar—Removal) process requires numerous applications, but due to the benign nature of the compounds used, this is not a problem.

The best mode contemplated is as follows: Soak wooden picks (finely whittled for difficult to reach, under the gum access) in one of many activators. Without limiting it to just these two activating liquids each having advantageous uses.

Mouthwash: gentle, antiseptic, easy on gums.

Amonia: has surprising nerve deadening properties.

Charcoal: Suffice it to say that pratically any type of finely powdered charcoal will work.

As the charcoal is freshly activated by collecting a small amount of powdered charcoal on the tip of the wooden or bamboo pick and applied.

As the pick gently skims and mixes the charcoal it soon becomes apparent that the tooth tartar is combining with the freshly activated charcoal and has taken on the appearance that matches sticky tar refered to here as tartar slag. The charcoal is quickly absorbed into the tartar turning a naturally dark substance even blacker, certainly identifying tartar deposits.

Now it becomes very much a matter of technique for removing the liguified sticky tartar slag. I assume that a dental vacumn would be very helpful, but for the self practitioner a toothbrush that has also had a small amount of freshly activated charcoal (dip into powder, dip into mouthwash) helps to remove and clean the tartar slag away from the teeth. A warm water rinse helps to clear the bits of the now deactivated charcoal away and allows the next application to go to work on the next layer of tartar. Fresh activator is essential to getting the most from each application. Contamination of the ends of the capturing devices and other removal aids will only slow the process.

As the tartar combines with the charcoal it is necessary to repeat the applications until all the tartar has been removed.

All tartar left on the teeth only becomes a superior anchor for future tartar.

This is the process as I understand it. Through this amazing step by step, layer by layer FACT-R process, the tartar that is the cause of many dental problems can be removed with these safe painfree and simple methods.

1. What I claim as my invention is the process of tooth tartar removal based on a self developed discovery of how tooth tartar can be liquified by freshly activated charcoal.
The hope is that this discovery and it's resultant process, including simple tool and removal devices, freshly activated charcoal two part toothpaste, charcoal tipped wooden picks, charcoal impregnated floss and the activators be combined into patentable processes and products under this FACT-R discovery.
